(19) United States
(12) Patent Application Publication
(10) Pub. No.: US 2026/0078334 A1
(43) Pub. Date: Mar. 19, 2026

(54) A METHOD FOR PRODUCING HEAT-KILLED LACTIC ACID BACTERIA WHICH HAVE AN IMPROVED ACTIVITY OF IMMUNE-REGULATION

(71) Applicant: CJ CHEILJEDANG CORPORATION, Seoul (KR)

(72) Inventors: Jae Seung PARK, Seoul (KR); Woo-yong JUNG, Seoul (KR); Hee Kyoung JUNG, Seoul (KR); Hyun Sun YUN, Seoul (KR); Jayoung LEE, Seoul (KR); Min-su KIM, Seoul (KR)

(73) Assignee: CJ CHEILJEDANG CORPORATION, Seoul (KR)

(21) Appl. No.: 19/164,542

(22) PCT Filed: Aug. 3, 2023
(86) PCT No.: PCT/KR2023/011409

§ 371 Date: Sep. 11, 2025

(30) Foreign Application Priority Data
Aug. 3, 2022 (KR) 10-2022-0096808

(51) Int. Cl. C12N 1/00; A61K 35/747; A61P 31/04; A61P 37/02; C12N 1/20; C12R 1/25

(52) U.S. Cl. CPC C12N 1/005; A61K 35/747; A61P 31/04; A61P 37/02; C12N 1/205; C12R 2001/25

(57) ABSTRACT

The present disclosure relates to: a method for preparing heat-killed lactic acid bacteria.

### TECHNICAL FIELD

The present disclosure relates to: a method for preparing heat-killed lactic acid bacteria, comprising a first heat treatment and a second heat treatment of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP; a method for enhancing an immunomodulatory ability of heat-killed lactic acid bacteria; a method for enhancing an ability of heat-killed lactic acid bacteria to produce interferon-gamma or reduce interleukin-4; a method for enhancing the ability to inhibit adhesion of harmful bacteria in the intestines; heat-killed lactic acid bacteria prepared by the method; and food, pharmaceutical, and feed compositions comprising the heat-killed lactic acid bacteria.

### BACKGROUND ART

Heat-killed lactic acid bacteria have various differences compared to live bacteria. Unlike live bacteria, heat-killed bacteria do not settle in the intestines to become active, but rather contribute to immunomodulation by coming into direct contact with immune cells. Since they are not live bacteria, they are not affected by the harsh environment (physical and chemical digestion processes) in the gastrointestinal tract and are heat-stable. Thus, they maintain certain functionalities during storage and delivery and can be applied in various ways to products that include a sterilization process. Due to such material stability characteristics, not only live bacteria but also heat-killed bacteria are being used in health functional foods, general foods, pharmaceuticals, animal feed, cosmetic raw materials, and the like (U.S. Pat. No. 8,361,481).

However, if live bacteria remain within heat-killed bacteria, live bacteria may cause changes in the quality of the applied product. Accordingly, active research on heat-killing processes is underway to maintain the quality of food and enhance its functionality while achieving the same immunomodulatory functions as those of conventional live bacterial products.

In particular, live bacteria can be killed by various methods. Physical and chemical methods including UV irradiation, high-pressure treatment, sonication, pulsed electric field sterilization, ohmic heating, supercritical carbon dioxide, drying, pH adjustment, acid dehydration activity, enzyme treatment, and solvent extraction have been reported.

However, the development of an efficient heat treatment method that maintains a capability of promoting host health equivalent to that of live bacteria while minimizing the impact on cellular structure remains limited.

### DISCLOSURE

### Technical Problem

The problem to be solved by the present disclosure is to provide: a method for preparing heat-killed lactic acid bacteria, comprising first heat treatment and second heat treatment steps of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP; heat-killed lactic acid bacteria prepared by the method; and food, pharmaceutical, and feed compositions comprising the bacteria.

### Technical Solution

It is one object of the present disclosure to provide a method for preparing heat-killed lactic acid bacteria, comprising first heat treatment and second heat treatment steps of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP.

It is one object of the present disclosure to provide a method for enhancing an immunomodulatory ability of heat-killed lactic acid bacteria, comprising first heat treatment and second heat treatment steps of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP.

It is one object of the present disclosure to provide a method for enhancing an ability of heat-killed lactic acid bacteria to produce interferon-gamma (IFN-γ) or to reduce interleukin-4 (IL-4), comprising first heat treatment and second heat treatment steps of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP.

It is one object of the present disclosure to provide a method for enhancing an ability of heat-killed lactic acid bacteria to inhibit adhesion of harmful bacteria in the intestines, comprising first heat treatment and second heat treatment steps of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP.

It is one object of the present disclosure to provide heat-killed lactic acid bacteria prepared by the method for preparing heat-killed lactic acid bacteria, the method for enhancing an immunomodulatory ability of heat-killed lactic acid bacteria, or the method for enhancing an ability of heat-killed lactic acid bacteria to produce IFN-γ or to reduce IL-4.

It is one object of the present disclosure to provide a food composition comprising the heat-killed lactic acid bacteria.

It is one object of the present disclosure to provide a pharmaceutical composition comprising the heat-killed lactic acid bacteria.

It is one object of the present disclosure to provide a feed composition comprising the heat-killed lactic acid bacteria.

It is one object of the present disclosure to provide use of the heat-killed lactic acid bacteria for immunomodulation, inhibiting adhesion of harmful bacteria in the intestines, increasing IFN-γ, and reducing IL-4.

### Advantageous Effects

The method for preparing heat-killed lactic acid bacteria according to the present disclosure can be used to prepare heat-killed bacteria with an immunomodulatory ability comparable to that of live bacteria and that exhibit an improved ability to inhibit adhesion of harmful bacteria in the intestines.

### BRIEF DESCRIPTION OF THE DRAWINGS

FIGS. 1 to 3 are diagrams illustrating the effect of heat-killed lactic acid bacteria CJLP133, CJLP243, and CJLP55 on increasing the production of interferon-gamma (IFN-γ).

FIGS. 4 to 6 are diagrams illustrating the effect of heat-killed lactic acid bacteria CJLP133, CJLP243, and CJLP55 on decreasing the production of interleukin-4 (IL-4).

FIGS. 7 to 9 are diagrams illustrating the effect of heat-killed lactic acid bacteria CJLP133, CJLP243, and CJLP55 on inhibiting the adhesion of harmful bacteria in the intestines.

FIG. 10 is a diagram showing the results of evaluating the production levels of IFN-γ and IL-4 in CJLP133 after a first heat treatment step according to the temperature and duration of the heat treatment.

FIG. 11 is a diagram showing the results of evaluating the production levels of IFN-γ and IL-4 in CJLP133 after a first or second heat treatment step.

FIG. 12 is a diagram showing the results of evaluating the production levels of IFN-γ and IL-4 in CJLP133 after first heat treatment step and second heat treatment step according to the temperature and duration of the heat treatments.

FIG. 13 is a diagram showing the results of evaluating the production levels of IFN-γ and IL-4 in CJLP243 after heat treatment according to the temperature and duration of the heat treatment.

FIG. 14 is a diagram showing the results of evaluating the production levels of IFN-γ and IL-4 in CJLP55 after heat treatment according to the temperature and duration of the heat treatment.

### DETAILED DESCRIPTION OF PREFERRED EMBODIMENTS

Hereinafter, the present disclosure will be described in detail. Meanwhile, with respect to common matters, each description and embodiment of an aspect disclosed herein can be applied to the description and embodiment of another aspect, respectively. Further, all combinations of various elements described herein fall within the scope of the present disclosure. In addition, the literature described herein may be incorporated by reference into the present disclosure. Moreover, the scope of the present disclosure is not limited by the specific description described below.

One aspect of the present disclosure provides a method for preparing heat-killed lactic acid bacteria, comprising:
a first heat treatment step of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP at a temperature of 40° C. or more and less than 60° C. for 6 to 56 hours; and
a second heat treatment step of heat-treating the lactic acid bacteria at a temperature of 90° C. to 125° C. for 10 minutes to 4 hours.

For the purposes of the present disclosure, the lactic acid bacteria belong to Lactobacillus plantarum, and specifically, are one or more selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP. Herein, unless otherwise specified, the term “lactic acid bacteria” refers to one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP.

The strains Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP used herein are strains deposited as disclosed in U.S. Pat. No. 10,130,666 B2 (EP 2457992 B1), U.S. Pat. No. 10,093,995 B2 (EP 2494031 B1), and U.S. Pat. No. 9,572,846 B2 (EP 2455450 B1), respectively.

As used herein, the term “Lactobacillus plantarum” refers to a microorganism of the genus Lactobacillus that is Gram-positive and produces lactic acid. It may be found in fermented foods including sauerkraut, pickles, and kimchi, but is not limited thereto.

As used herein, the term “heat-killed lactic acid bacteria” refers to a form in which bacterial growth is inhibited through heat-killing of live bacteria. The heat-killed lactic acid bacteria undergo changes in some or all of the activities originally present in the live bacteria and may exhibit increased stability during storage or within the host compared to the live bacteria.

The heat-killed lactic acid bacteria of the present disclosure may be present in the form of a culture, a fermented product, or a culture medium thereof; or as a dilution, a concentrate, a dried product, a freeze-dried product, or a lysate of one or more of the foregoing. It may also include a culture in which live bacteria in a live culture are heat-killed, and the form of the culture is not particularly limited as long as it comprises heat-killed lactic acid bacteria.

According to the type of lactic acid bacteria, the specific method for preparing the heat-killed lactic acid bacteria, and conditions thereof, the method and conditions have significant differences on the ability of the heat-killed lactic acid bacteria to inhibit adhesion of harmful bacteria in the intestines, the cellular structure, and the effect on the host. In particular, when preparing heat-killed lactic acid bacteria by heat treatment, it is important to use the conditions, steps, etc., required for each bacterial strain in order for the heat-killed lactic acid bacteria to exhibit abilities equivalent to those of live bacteria. Accordingly, the method for preparing heat-killed bacteria by heat treatment must be specifically selected depending on the bacterial strain and the intended effect, and the significance of the present application lies in providing a method for preparing heat-killed lactic acid bacteria specifically optimized for Lactobacillus plantarum CJLP133, CJLP243, and CJLP55.

In one embodiment, when heat-killed lactic acid bacteria are prepared by the method comprising a first heat treatment step and a second heat treatment step of the present disclosure, the heat-killed lactic acid bacteria of Lactobacillus plantarum CJLP133, CJLP243, and CJLP55 may inhibit adhesion of harmful bacteria in the intestines while enhancing the immunomodulatory ability of the host to a level comparable to that of the live bacteria of the strains.

The first heat treatment step of the present disclosure is heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP at a temperature of 40° C. or more and less than 60° C. for 6 to 56 hours.

As used herein, the term “heat treatment” refers to applying heat to lactic acid bacterial cells. The heat treatment is not limited to a specific method such as hot air or heating. While general culturing is conducted in the presence of sugars used as a carbon source and other nutrients to allow microbial growth, the first heat treatment and second heat treatment of the present disclosure may be heat treatments performed on microbial cells obtained by centrifugation or the like. The heat treatment of the present disclosure may be performed in the presence of distilled water or the like; however, unlike general culturing, it may be distinguished from culturing in that heat is applied in the absence of sugars and nutrients.

In one embodiment, the heat treatment may be performed after obtaining microbial cells by a centrifugation step during the stationary phase of the culturing step, in which microbial growth is inhibited due to the depletion of sugars in the culture medium, and mixing with sterile distilled water without any nutrients; however, the heat treatment is not limited thereto.

The temperature of the first heat treatment step may be 40° C. or more and less than 60° C., and specifically, 41° C. or more and less than 60° C., 42° C. or more and less than 60° C., 43° C. or more and less than 60° C., 44° C. or more and less than 60° C., 45° C. or more and less than 60° C., 46° C. or more and less than 60° C., 47° C. or more and less than 60° C., 48° C. or more and less than 60° C., 49° C. or more and less than 60° C., 50° C. or more and less than 60° C., 41° C. to 50° C., 42° C. to 50° C., 43° C. to 50° C., 44° C. to 50° C., 45° C. to 50° C., 46° C. to 50° C., 47° C. to 50° C., 48° C. to 50° C., 49° C. to 50° C., 40° C. to 49° C., 40° C. to 48° C., 40° C. to 47° C., 40° C. to 46° C., 40° C. to 45° C., 40° C. to 44° C., 40° C. to 43° C., 40° C. to 42° C., 40° C. to 41° C., or 40° C., but is not limited thereto.

The temperature of the first heat treatment step may be a temperature capable of inhibiting the adhesion of harmful bacteria in the intestines while enhancing the immunomodulatory ability of the heat-killed bacteria to a level comparable to that of the live bacteria. When the heat treatment is performed at a temperature of less than 40° C. or more than 60° C., the heat may not be transferred (e.g., cooling), the cellular structure of the live bacteria may be affected, or the immunomodulatory ability of the heat-killed bacteria and/or the ability to inhibit the adhesion of harmful bacteria in the intestines may be reduced compared to that of the live bacteria.

The duration of the first heat treatment step may be 6 to 56 hours, and specifically, 8 to 56 hours, 10 to 56 hours, 12 to 56 hours, 6 to 48 hours, 8 to 48 hours, 10 to 48 hours, 12 to 48 hours, 6 to 36 hours, 8 to 36 hours, 10 to 36 hours, 12 to 36 hours, 6 to 24 hours, 8 to 24 hours, 10 to 24 hours, 12 to 24 hours, 6 to 12 hours, 8 to 12 hours, 10 to 12 hours, or 12 hours, but is not limited thereto.

The duration of the first heat treatment step may be a duration capable of enhancing the immunomodulatory ability of the heat-killed bacteria to a level comparable to that of the live bacteria and/or inhibiting the adhesion of harmful bacteria in the intestines at the temperature of the first heat treatment step. When the heat treatment is performed for less than 6 hours, the immunomodulatory ability of the heat-killed bacteria or the ability to inhibit the adhesion of harmful bacteria in the intestines may be decreased compared to that of the live bacteria.

However, even when the duration of the first heat treatment step of the present disclosure is 12 hours or more, the immunomodulatory ability is enhanced to a level comparable to that after a first heat treatment for 12 hours. Accordingly, a duration of 12 hours, as well as a duration of 56 hours or more, may be included in the present disclosure.

The second heat treatment step of the present disclosure is heat-treating the lactic acid bacteria after first heat treatment at a temperature of 90° C. to 125° C. for 10 minutes to 4 hours.

The temperature of the second heat treatment step may be 90° C. to 125° C., and specifically, 95° C. to 125° C., 100° C. to 125° C., 105° C. to 125° C., 110° C. to 125° C., 115° C. to 125° C., 120° C. to 125° C., 90° C. to 121° C., 95° C. to 121° C., 100° C. to 121° C., 105° C. to 121° C., 110° C. to 121° C., 115° C. to 121° C., 120° C. to 121° C., 90° C. to 120° C., 95° C. to 120° C., 100° C. to 120° C., 105° C. to 120° C., 110° C. to 120° C., 115° C. to 120° C., 90° C. to 115° C., 95° C. to 115° C., 100° C. to 115° C., 105° C. to 115° C., 110° C. to 115° C., 90° C. to 110° C., 95° C. to 110° C., 100° C. to 110° C., 105° C. to 110° C., 90° C. to 105° C., 95° C. to 105° C., 100° C. to 105° C., 90° C. to 100° C., 95° C. to 100° C., or 100° C., but is not limited thereto.

The temperature of the second heat treatment step may be a temperature capable of reducing the number of live bacteria while simultaneously maintaining the immunomodulatory ability of the heat-killed bacteria or the ability to inhibit adhesion of harmful bacteria in the intestines to a level comparable to that of the live bacteria or to a level comparable to that of the bacteria after performing the first heat treatment step only. When the heat treatment is performed at a temperature of less than 90° C. or more than 125° C., the cellular structure of the live bacteria may be affected, the immunomodulatory ability of the heat-killed bacteria and/or the ability to inhibit the adhesion of harmful bacteria in the intestines may be reduced compared to those of the live bacteria, or a high number of live bacteria may remain.

The duration of the second heat treatment step may be 10 minutes to 4 hours, and specifically, 20 minutes to 4 hours, 30 minutes to 4 hours, 40 minutes to 4 hours, 50 minutes to 4 hours, 1 hour to 4 hours, 1 hour 30 minutes to 4 hours, 2 hours to 4 hours, 2 hours 30 minutes to 4 hours, 3 hours to 4 hours, 10 minutes to 3 hours, 20 minutes to 3 hours, 30 minutes to 3 hours, 40 minutes to 3 hours, 50 minutes to 3 hours, 1 hour to 3 hours, 1 hour 30 minutes to 3 hours, 2 hours to 3 hours, 2 hours 30 minutes to 3 hours, 10 minutes to 2 hours, 20 minutes to 2 hours, 30 minutes to 2 hours, 40 minutes to 2 hours, 50 minutes to 2 hours, 1 to 2 hours, 1 hour and 30 minutes to 2 hours, 1 hour, or 2 hours, but is not limited thereto.

The duration of the second heat treatment step may be a duration capable of reducing the number of live bacteria at the temperature of the second heat treatment step while maintaining the immunomodulatory ability of the heat-killed bacteria or the ability to inhibit adhesion of harmful bacteria in the intestines to a level comparable to that of the live bacteria or to a level comparable to that of the bacteria after performing the first heat treatment step only. When the heat treatment is performed for less than 10 minutes or more than 4 hours, the cellular structure of the live bacteria may be affected, the immunomodulatory ability of the heat-killed bacteria and/or the ability to inhibit the adhesion of harmful bacteria in the intestines may be significantly reduced compared to those of the live bacteria, or a high number of live bacteria may remain.

The heat treatment (first and second heat treatments) of the present disclosure may be performed only on lactic acid bacterial cells separated from a culture solution, or may be performed on a culture comprising the lactic acid bacterial cells, a fermented product, a culture medium of the lactic acid bacteria, or a dilution, a concentrate, a dried product, a lysate, etc., of one or more of the foregoing, but is not limited as long as it comprises live lactic acid bacteria.

The heat treatment of the present disclosure is not limited to specific methods as long as heat is applied, and a person skilled in the art may appropriately select a heat treatment method, provided that the temperature and duration defined in the present disclosure are satisfied.

For preparation efficiency, any pre-treatment or additional conditions may be added before or after a heat treatment step for preparing heat-killed bacteria of the present disclosure. For example, distilled water may be introduced to and/or mixed with the live bacterial cells before the heat treatment.

The heat-killed bacterial cells may undergo any additional process for the preparation of a heat-killed formulation, which includes, for example, concentration, drying, or fermentation. The concentration, drying, fermentation, etc. may be carried out by any method commonly used for lactic acid bacteria in the art, without limitation as to the type thereof; however, the drying may be performed, for example, by thermal (hot air) drying or freeze-drying.

In one embodiment, while general culturing is conducted in the presence of sugars used as a carbon source and other nutrients to allow microbial growth, the first heat treatment step and second heat treatment step of the present disclosure may be heat treatment performed after obtaining microbial cells by centrifugation during the stationary phase, in which growth is inhibited due to the depletion of sugars in the culture medium, and mixing with sterile distilled water without any nutrients.

In one embodiment, the method for preparing heat-killed lactic acid bacteria of the present disclosure may further comprise, prior to the first heat treatment step, one or more steps selected from the group consisting of: culturing one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP (hereinafter, referred to as “the culturing step”); centrifuging the lactic acid bacteria in a stationary phase (hereinafter, referred to as “the centrifugation step”); and injecting sterilized distilled water to the lactic acid bacterial cells obtained from the centrifugation in an amount of 0.75 to 1.25 times the amount of cells (hereinafter, referred to as “the sterilized distilled water injection step”). However, the method is not limited thereto, and the amount of the sterilized distilled water is not limited to the range of 0.75 to 1.25 times.

As used herein, the term “culturing” refers to growing Lactobacillus plantarum of the present disclosure in the presence of sugars used as a carbon source and other nutrients under properly controlled environmental conditions. Herein, the culturing process may be performed in a suitable medium known in the art under suitable culturing conditions known in the art. Such a culturing process may be readily adjusted and used by a person skilled in the art according to the selected strain. Specifically, the culturing may be batch culturing, continuous culturing, and/or fed-batch culturing, but is not limited thereto.

Lactobacillus plantarum of the present disclosure may be cultured in a common medium containing suitable carbon sources, nitrogen sources, phosphorus sources, inorganic compounds, amino acids, and/or vitamins, etc., with the temperature, pH, etc. controlled and under facultative anaerobic or anaerobic conditions.

During the culturing of the present disclosure, the temperature may be maintained at 20° C. to 38° C., specifically 21° C. to 38° C., 23° C. to 38° C., 25° C. to 38° C., 27° C. to 38° C., 30° C. to 38° C., 21° C. to 35° C., 23° C. to 35° C., 25° C. to 35° C., 27° C. to 35° C., or 30° C. to 35° C., and the culturing may be performed for about 10 to 160 hours, about 20 to 130 hours, about 24 to 120 hours, about 36 to 120 hours, about 48 to 120 hours, about 48 hours or more, about 48 hours, about 72 hours, or about 120 hours, but the temperature and duration are not limited thereto.

The culturing may be performed until the stationary phase of the microbial growth phase, but is not limited thereto.

The growth curve of a microorganism is a graph that shows the growth process, in which the number of microorganisms increases, by plotting the logarithmic value of the bacterial count over the duration of the culturing. It is also referred to as a microbial proliferation curve and shows the degree of microbial proliferation or growth over the duration of the culturing. The growth phase of a microorganism is classified into a lag phase, a log (exponential) phase, a stationary phase, and a death phase, and the characteristics of each phase are as follows:

Lag phase: a preparatory period during which the microorganism synthesizes substances necessary for proliferation in response to new nutrients and a new environment after being inoculated into a medium. The cell count may not increase significantly during the lag phase.

Log phase: a period during which the microorganism undergoes rapid cell division at its maximum rate after adapting to the culture environment and completing the preparatory steps for proliferation. During the log phase, the microorganism can grow and divide at the maximum rate permitted by their genetic characteristics, the composition of the medium, and the culturing conditions. As microorganisms grow rapidly, the supplied nutrients are rapidly consumed, and microbial metabolites and growth inhibitory substances may be produced.

Stationary phase: a period in the growth phase, in which the number of viable cells reaches its maximum. The number of viable cells may not increase or decrease, as microbial cell division and death reach an equilibrium. It may be a period in which exponential growth declines due to factors that limit population growth, such as nutrient depletion, accumulation of growth inhibitory substances, or lack of space.

Death phase: a period in which the number of heat-killed bacteria increases rapidly and the number of live bacteria decreases. The death phase may be a period in which the equilibrium between microbial cell division and death is disrupted due to energy depletion, destruction of cellular structure, decomposition of cell components, and inactivation of enzymes.

The culturing of the present disclosure may be culturing during which no additional medium is added after the addition of the medium at the start of the cultivation. Specifically, batch culture may be used, but the culturing is not limited thereto. Accordingly, cells may be obtained by centrifugation during the stationary phase in which the added sugars, i.e., nutrients, are depleted as microorganism growth progresses and exponential growth decreases.

The centrifugation step may be a step for obtaining lactic acid bacterial cells.

The centrifugation may be performed at 10,000 RPM to 15,000 RPM, 11,000 RPM to 15,000 RPM, 12,000 RPM to 15,000 RPM, 13,000 RPM to 15,000 RPM, 14,000 RPM to 15,000 RPM, 10,000 RPM to 14,000 RPM, 11,000 RPM to 14,000 RPM, 12,000 RPM to 14,000 RPM, 13,000 RPM to 14,000 RPM, 10,000 RPM to 13,000 RPM, 11,000 RPM to 13,000 RPM, 12,000 RPM to 13,000 RPM, 10,000 RPM to 12,000 RPM, 11,000 RPM to 12,000 RPM, 10,000 RPM to 11,000 RPM, or 15,000 RPM, but is not limited thereto.

The sterilized distilled water injection step may involve injecting sterilized distilled water to the lactic acid bacterial cells in an amount of 0.75 to 1.25 times, 0.75 to 1.2 times, 0.75 to 1.15 times, 0.75 to 1.1 times, 0.75 to 1.0 times, 0.75 to 0.95 times, 0.75 to 0.9 times, 0.75 to 0.85 times, 0.75 to 0.8 times, 0.8 to 1.25 times, 0.8 to 1.2 times, 0.8 to 1.15 times, 0.8 to 1.1 times, 0.8 to 1.0 times, 0.8 to 0.95 times, 0.8 to 0.9 times, 0.8 to 0.85 times, 0.85 to 1.25 times, 0.85 to 1.2 times, 0.85 to 1.15 times, 0.85 to 1.1 times, 0.85 to 1.0 times, 0.85 to 0.95 times, 0.85 to 0.9 times, 0.9 to 1.25 times, 0.9 to 1.2 times, 0.9 to 1.15 times, 0.9 to 1.1 times, 0.9 to 1.0 times, 0.9 to 0.95 times, 0.95 to 1.25 times, 0.95 to 1.2 times, 0.95 to 1.15 times, 0.95 to 1.1 times, 0.95 to 1.0 times, 1.0 to 1.25 times, 1.0 to 1.2 times, 1.0 to 1.15 times, 1.0 to 1.1 times, 1.1 to 1.25 times, 1.1 to 1.2 times, or 1.1 to 1.15 times the amount of the bacterial cells, but is not limited thereto.

The sterilized distilled water injection step may further comprise mixing the injected sterilized distilled water with the lactic acid bacterial cells (hereinafter, referred to as “the mixing step”), but is not limited thereto.

As used herein, the term “immunomodulatory” or “immunomodulation” means modulating immunity to the benefit of an individual, such as activating immune cells, increasing cytokine secretion, promoting immune function, and maintaining balance in immune cell activity. In one embodiment, immunomodulation may be an enhancement of immunity.

Among the immunity, immunity involving T lymphocytes, which are central to adaptive immunity, can be divided into Th1 responses, which are cellular immunity, and TH2 responses, which are humoral immunity. Through Th1 responses, interferon (IFN), IL-12, IL-18, etc. can be produced, and IL-4, IL-10, PGE2, etc. can be produced through TH2 responses. When any one of the Th1 and TH2 responses is excessive or insufficient, diseases resulting from immune dysregulation may occur.

In one embodiment, the enhancement of immunity may be one or more selected from the group consisting of an ability to promote IFN-γ, a decrease in IL-4 production, and the maintenance or enhancement of the balance between Th1 and TH2 responses, but is not limited thereto.

In one embodiment, the method comprising the first heat treatment step and second heat treatment step of the present disclosure may enhance the immunomodulatory ability of the heat-killed lactic acid bacteria, but is not limited thereto.

In one embodiment, the method of the present disclosure comprising the first heat treatment step and second heat treatment step may, compared to that comprising the first heat treatment or the second heat treatment alone, reduce the number of live bacteria while enhancing the immunomodulatory ability of the heat-killed bacteria to a level comparable to that of the live bacteria, inhibiting the adhesion of harmful bacteria in the intestines, and/or maintaining the balance of intestinal bacteria, but is not limited thereto.

In one embodiment, the method of the present disclosure comprising the first heat treatment step and second heat treatment step may enhance the ability of the heat-killed lactic acid bacteria to promote IFN-γ production, to reduce IL-4 production, to inhibit adhesion of harmful bacteria in the intestines, or a combination thereof, but is not limited thereto.

In one embodiment, the method of the present disclosure comprising the first heat treatment step and second heat treatment step may maintain or enhance the balance between the Th1 and TH2 responses in a subject (host) to which the heat-killed lactic acid bacteria prepared by the first heat treatment step and second heat treatment step have been administered, but is not limited thereto.

In one embodiment, the method of the present disclosure comprising the first heat treatment step and second heat treatment step may prevent diseases resulting from the imbalance between the Th1 and Th2 responses in a subject to which the heat-killed lactic acid bacteria prepared by the first heat treatment and second heat treatment steps have been administered, but is not limited thereto.

One aspect of the present disclosure provides a method for enhancing an immunomodulatory ability of heat-killed lactic acid bacteria, comprising: a first heat treatment step of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP at a temperature of 40° C. or more and less than 60° C. for 6 to 56 hours; and
a second heat treatment step of heat-treating the lactic acid bacteria at a temperature of 90° C. to 125° C. for 10 minutes to 4 hours.

The terms “lactic acid bacteria”, “first heat treatment”, “second heat treatment”, “heat-killed lactic acid bacteria”, “culture”, “immunomodulation”, etc. are as described in other aspects, and the culturing step, centrifugation step, sterilized distilled water injection step, mixing step, etc. can also be similarly applied to the method for enhancing an immunomodulatory ability of heat-killed lactic acid bacteria of the present disclosure.

One aspect of the present disclosure provides a method for enhancing an ability of heat-killed lactic acid bacteria to produce IFN-γ or to reduce IL-4, comprising: a first heat treatment step of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP at a temperature of 40° C. or more and less than 60° C. for 6 to 56 hours; and
a second heat treatment step of heat-treating lactic acid bacteria of a Lactobacillus sp. at a temperature of 90° C. to 125° C. for 10 minutes to 4 hours.

The terms “lactic acid bacteria”, “first heat treatment”, “second heat treatment”, “heat-killed lactic acid bacteria”, “culture”, “immunomodulation”, etc. are as described in other aspects, and the culturing step, centrifugation step, sterilized distilled water injection step, mixing step, etc. can also be similarly applied to the method for enhancing an ability to produce IFN-γ or to reduce IL-4 of the present disclosure.

One aspect of the present disclosure provides a method for enhancing an ability of heat-killed lactic acid bacteria to inhibit adhesion of harmful bacteria in the intestines, comprising: a first heat treatment step of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP at a temperature of 40° C. or more and less than 60° C. for 6 to 56 hours; and
a second heat treatment step of heat-treating lactic acid bacteria of a Lactobacillus sp. at a temperature of 90° C. to 125° C. for 10 minutes to 4 hours.

The terms “lactic acid bacteria”, “first heat treatment”, “second heat treatment”, “heat-killed lactic acid bacteria”, “culture”, “immunomodulation”, etc. are as described in other aspects, and the culturing step, centrifugation step, sterilized distilled water injection step, mixing step, etc. can also be similarly applied to the method for enhancing an ability of heat-killed lactic acid bacteria to inhibit adhesion of harmful bacteria in the intestines of the present disclosure.

As used herein, the term “harmful bacteria” refers to bacteria that pose adverse effects on a subject. The “harmful bacteria” may be a microorganism of Clostridium sp., Shigella sp., Salmonella sp., Vibrio sp., Yersinia sp., and/or Staphylococcus sp., specifically Staphylococcus aureus, and/or Escherichia coli, and more specifically enterotoxigenic Escherichia coli, but is not limited thereto.

As used herein, the term “inhibiting adhesion of harmful bacteria in the intestines” refers to reducing the adhesion of the above-described harmful bacteria in the intestines.

One aspect of the present disclosure provides heat-killed lactic acid bacteria prepared by the method for preparing heat-killed lactic acid bacteria, the method for enhancing an immunomodulatory ability of heat-killed lactic acid bacteria, the method for enhancing an ability of heat-killed lactic acid bacteria to inhibit adhesion of harmful bacteria in the intestines, or the method for enhancing an ability of heat-killed lactic acid bacteria to produce IFN-γ or to reduce IL-4.

The terms “lactic acid bacteria”, “heat-killed lactic acid bacteria”, “immunomodulation”, etc. are as described in other aspects.

The heat-killed lactic acid bacteria of the present disclosure may be included in a food composition, a pharmaceutical composition, or a non-pharmaceutical composition, and the composition may be for immunomodulation.

One aspect of the present disclosure provides a food composition comprising heat-killed lactic acid bacteria prepared by the method for preparing heat-killed lactic acid bacteria, the method for enhancing an immunomodulatory ability of heat-killed lactic acid bacteria, the method for enhancing an ability of heat-killed lactic acid bacteria to inhibit adhesion of harmful bacteria in the intestines, or the method for enhancing an ability of heat-killed lactic acid bacteria to produce IFN-γ or to reduce IL-4.

The terms “lactic acid bacteria”, “heat-killed lactic acid bacteria”, “immunomodulation”, etc. are as described in other aspects. In one embodiment, the food composition may be for immunomodulation, but is not limited thereto.

The food composition may be in the form of a health functional food or a food additive.

As used herein, the term “health functional food” refers to a food that is manufactured and processed using raw materials or ingredients having functionality beneficial to the human body (Article 3, Paragraph 1), and the term “functionality” refers to controlling nutrients for the structure or functions of the human body or providing beneficial effects for health purposes, such as physiological actions (Article 3 Paragraph 2), in accordance with the Health Functional Food Act.

The food composition of the present disclosure may further comprise a food additive, and the suitability thereof as a “food additive”, unless otherwise specified, is determined according to the standards and criteria for the corresponding item in the General Rules and General Test Methods of the Food Additives Code approved by the Ministry of Food and Drug Safety.

Items listed in the “Food Additives Code” include, for example, chemical synthetic products such as ketones, glycine, potassium citrate, nicotinic acid, and cinnamic acid; natural additives such as persimmon pigment, licorice root extract, crystalline cellulose, and guar gum; and mixed preparations such as a sodium L-glutamate preparation, an alkali agent for addition to noodles, a preservative preparation, and a tar color preparation.

Examples of food comprising the active ingredient of the present disclosure may include confectionery, such as bread, rice cakes, dried snacks, candies, chocolates, chewing gum, and jams; ice cream products, such as ice creams, frozen desserts, and ice cream powder; dairy products, such as milk, low-fat milk, lactose-free milk, processed milk, goat milk, fermented milk, buttermilk, concentrated milk, milk creams, natural cheese, processed cheese, powdered milk, and whey; meat products, such as processed meat products, processed egg products, and hamburgers; fish meat products, such as fish cakes and fish meat processed products, for example, ham, sausage, and bacon; noodles, such as ramen, dried noodles, fresh noodles, instant fried noodles, gelatinized dried noodles, modified cooked noodles, frozen noodles, and pastas; beverages, such as fruit beverages, vegetable beverages, carbonated drinks, soybean milk, probiotic drinks including yogurts, and mixed beverages; seasoning foods, such as soy sauce, fermented bean paste, red pepper paste, black bean paste, fast-fermented bean paste, mixed paste, vinegar, sauces, tomato ketchup, curry, and dressings; and fermented foods, but are not limited thereto.

In addition to the above, the food composition of the present disclosure may comprise various nutrients, vitamins, electrolytes, flavoring agents, colorants, pectic acid and salts thereof, alginic acid and salts thereof, organic acids, protective colloid thickeners, pH adjusters, stabilizers, preservatives, glycerin, alcohols, carbonating agents used in carbonated drinks, and the like. Moreover, the food composition of the present disclosure may comprise fruit pulp for manufacture of natural fruit juices, fruit juice beverages, and vegetable beverages. These ingredients may be used alone or in combination.

Meanwhile, examples of carriers, excipients, and diluents suitable for food formulation include crystalline glucose, maltodextrin, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oils, but are not limited thereto. In addition, fillers, anti-coagulants, lubricants, humectants, flavoring agents, and antiseptics may be further included.

One aspect of the present disclosure provides a pharmaceutical composition comprising heat-killed lactic acid bacteria prepared by the method for preparing heat-killed lactic acid bacteria, the method for enhancing an immunomodulatory ability of heat-killed lactic acid bacteria, the method for enhancing an ability of heat-killed lactic acid bacteria to inhibit adhesion of harmful bacteria in the intestines, or the method for enhancing an ability of heat-killed lactic acid bacteria to produce IFN-γ or to reduce IL-4.

The terms “lactic acid bacteria”, “heat-killed lactic acid bacteria”, and “immunomodulation” are as described in other aspects. In one embodiment, the pharmaceutical composition may be for immunomodulation, but is not limited thereto.

The pharmaceutical composition of the present disclosure may be administered via any general route as long as the composition can reach a target tissue. Various modes of administration including intraperitoneal administration, oral administration, enteral administration (e.g., rectal administration), intravenous administration, intramuscular administration, subcutaneous administration, intradermal administration, topical administration, intranasal administration, and intrapulmonary administration may be considered, but the present disclosure is not limited to such exemplary modes of administration.

The pharmaceutical composition of the present disclosure may further comprise a pharmaceutically acceptable carrier. For oral administration, the pharmaceutically acceptable carrier may include binders, lubricants, disintegrants, excipients, solubilizers, dispersants, stabilizers, suspending agents, coloring agents, and flavoring agents. For an injection, the pharmaceutically acceptable carrier may include buffers, preservatives, analgesics, solubilizers, isotonic agents, and stabilizers. For topical administration, examples of the pharmaceutically acceptable carrier may include bases, excipients, lubricants, and preservatives. The pharmaceutical composition of the present disclosure may be formulated into various dosage forms in combination with the above-described pharmaceutically acceptable carriers. For example, for oral administration, the pharmaceutical composition may be formulated into a tablet, troche, capsule, elixir, suspension, syrup, or wafer. For an injection, the pharmaceutical composition may be formulated into an ampule as a single-dose formulation or a multiple-dose formulation, such as a multi-dose container. The composition may also be formulated into a solution, suspension, tablet, pill, capsule, and sustained-release preparation.

Meanwhile, examples of carriers, excipients, and diluents suitable for formulation include crystalline glucose, maltodextrin, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oils, but are not limited thereto. In addition, fillers, anti-coagulants, lubricants, humectants, flavoring agents, and antiseptics may be further included.

The pharmaceutical composition of the present disclosure may be administered in a pharmaceutically effective amount.

As used herein, the term “pharmaceutically effective amount” refers to an amount that is sufficient to treat a disease at a reasonable benefit/risk ratio applicable to medical treatment, and the level of the effective dose may be determined depending on factors including the subject type, severity of disease, age, sex, type of disease, activity of the drug, drug sensitivity, time of administration, route of administration, excretion rate, duration of treatment, and concurrently used drugs, as well as other factors well known in the medical field. The pharmaceutical composition of the present disclosure may be administered as a stand-alone treatment or in combination with other treatments, and may be administered sequentially or simultaneously with conventional treatments. Additionally, it may be administered as a single dose or multiple doses. It is important to administer the minimal effective amount that maximizes efficacy without causing side effects while taking all of the above elements into account, and the amount may be readily determined by a person skilled in the art. The preferred dosage of the pharmaceutical composition of the present disclosure may be determined by several relevant factors including the required degree of immunomodulation, the route of administration, the age, sex, and weight of the patient, and the severity of the disease.

The pharmaceutical composition of the present disclosure may be co-administered with an immunomodulator. In particular, the dose of the co-administered immunomodulator may be reduced, thereby reducing side effects of the immunomodulator and enhancing the therapeutic compliance of a patient. The administration may be carried out once a day or in multiple divided doses.

The pharmaceutical composition of the present disclosure can be used not only in the form of a pharmaceutical for humans but also in the form of a veterinary pharmaceutical. Herein, the animal includes livestock and pets.

It is one object of the present disclosure to provide a feed composition comprising heat-killed lactic acid bacteria prepared by the method for preparing heat-killed lactic acid bacteria, the method for enhancing an immunomodulatory ability of heat-killed lactic acid bacteria, the method for enhancing an ability of heat-killed lactic acid bacteria to inhibit adhesion of harmful bacteria in the intestines, or the method for enhancing an ability of heat-killed lactic acid bacteria to produce IFN-γ or to reduce IL-4.

The terms “lactic acid bacteria”, “heat-killed lactic acid bacteria”, “immunomodulation”, etc. are as described in other aspects. In one embodiment, the feed composition may be for immunomodulation, but is not limited thereto.

The feed composition may be in the form of a feed additive composition in addition to a feed composition.

When the composition is prepared as a feed additive, the composition may be prepared as a highly concentrated solution of 20% to 90% or in the form of a powder or granules. The feed additive may further comprise any one or more of organic acids such as citric acid, fumaric acid, adipic acid, lactic acid, and malic acid; phosphate salts such as sodium phosphate, potassium phosphate, acidic pyrophosphate, and polyphosphates; or natural anti-oxidants such as polyphenols, catechin, alpha-tocopherol, rosemary extract, vitamin C, green tea extract, licorice extract, chitosan, tannic acid, and phytic acid. When prepared as a feed, the composition may be formulated into a common feed form and may include conventional feed ingredients.

The feed and feed additive may further comprise grains, for example, powdered or crushed wheat, oat, barley, corn, and rice; plant protein feeds, for example, feeds with rape, bean and sunflower as main ingredients; animal protein feeds, for example, powdered blood, powdered meat, powdered bone, and powdered fish; and sugars and dairy products, for example, dried components including various types of powdered milk and powdered whey. In addition to the above, the feed and feed additive may further comprise nutritional supplements, digestion and absorption enhancers, growth accelerators, and the like.

The feed additive may be administered to the animal alone or in combination with other feed additives in edible carriers. Further, the feed additive may readily be administered to the animal as a top dressing, by directly mixing with an animal feed, or as an oral formulation separately from the feed. When the feed additive is administered separately from an animal feed, the feed additive may be prepared as an immediate-release or sustained-release formulation in combination with a pharmaceutically acceptable edible carrier well known in the art. The edible carrier may be solid or liquid and may include, for example, corn starch, lactose, sucrose, soybean flakes, peanut oil, olive oil, sesame oil, and propylene glycol. When a solid carrier is used, the feed additive may be a top dressing in the form of tablets, capsules, powders, troches, sugar-coated tablets, or non-dispersible forms. When a liquid carrier is used, the feed additive may be in the form of soft gelatin capsules, syrups, suspensions, emulsions, or solutions.

The feed and feed additive may contain adjuvants, such as preservatives, stabilizing agents, humectants, emulsifiers, and solution promoters. The feed additive may be added to animal feed for use by immersion, spraying, or mixing.

Meanwhile, examples of carriers, excipients, and diluents suitable for feed or feed additive formulation include crystalline glucose, maltodextrin, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oils, but are not limited thereto. In addition, fillers, anti-coagulants, lubricants, humectants, flavoring agents, and antiseptics may be further included.

The feed or feed additive of the present disclosure may be applied to the diet of numerous animals, including mammals, poultry, and fish.

The feed may be used for mammals such as pigs, cows, horses, sheep, rabbits, goats, rodents, and laboratory rodents such as rats, hamsters, and guinea pigs, as well as companion animals (e.g., dogs and cats), for poultry, such as chickens, turkeys, ducks, geese, pheasants, and quails, and for fish such as carp, crucian carp, and trout. However, the animals are not limited thereto.

One aspect of the present disclosure provides an immunomodulation method comprising administering, to a subject, heat-killed lactic acid bacteria prepared by the method for preparing heat-killed lactic acid bacteria, the method for enhancing an immunomodulatory ability of heat-killed lactic acid bacteria, the method for enhancing an ability of heat-killed lactic acid bacteria to inhibit adhesion of harmful bacteria in the intestines, or the method for enhancing an ability of heat-killed lactic acid bacteria to produce IFN-γ or to reduce IL-4; or a composition comprising the same.

The terms “lactic acid bacteria”, “heat-killed lactic acid bacteria”, “immunomodulation”, etc. are as described in other aspects.

The immunomodulation method of the present disclosure comprises administering the composition to a subject in need of immunomodulation in a food-effective, pharmaceutically effective, or feed-effective amount. The subject refers to all mammals including dogs, cows, horses, rabbits, mice, rats, chickens, and humans, but the mammals of the present disclosure are not limited thereto. The composition may be administered via parenteral, subcutaneous, intraperitoneal, intrapulmonary, and intranasal routes. For local treatment, it may be administered by any suitable method, including intralesional administration, if necessary. Parenteral administration includes intramuscular, intravenous, intra-arterial, intraperitoneal, or subcutaneous administration. The dosage form may be tablets, troches, capsules, elixirs, suspensions, syrups, wafers, intravenous injections, subcutaneous injections, intradermal injections, intramuscular injections, or drip injections for oral administration, but is not limited thereto. The preferred dosage of the pharmaceutical composition of the present disclosure may vary depending on the condition and weight of the subject, the severity of disease, the form of drug, and the route and duration of administration, and may be appropriately determined by a person skilled in the art.

One aspect of the present disclosure provides use of the heat-killed lactic acid bacteria prepared by the method for preparing heat-killed lactic acid bacteria, the method for enhancing an immunomodulatory ability of heat-killed lactic acid bacteria, or the method for enhancing an ability of heat-killed lactic acid bacteria to produce IFN-γ or to reduce IL-4 for immunomodulation; inhibiting adhesion of harmful bacteria in the intestines; increasing IFN-γ; and reducing IL-4 of the present disclosure.

The terms “lactic acid bacteria”, “heat-killed lactic acid bacteria”, “immunomodulation”, etc. are as described in other aspects.

### MODE FOR CARRYING OUT THE INVENTION

The present disclosure will be described in detail by way of Examples. However, these Examples are given for illustrative purposes only, and the scope of the invention is not intended to be limited by these Examples. Meanwhile, the technical descriptions absent in the present disclosure may be sufficiently understood and readily practiced by a person of ordinary skill in the art of the present disclosure or related art.

### Example 1: Production and Recovery of Heat-Killed Lactic Acid Bacteria

A culture solution was prepared by inoculating a Lactobacillus plantarum strain into an edible medium and culturing at 37° C. until all the added sugar was consumed. When maximum microbial growth was reached during the stationary phase, the cells were obtained by centrifugation at 15,000 RPM. Sterile distilled water was added and mixed with the obtained cells in an amount equal to the weight of the cells. The mixture was placed back into the incubator, heated to 40° C., and then heat-treated for 12 hours. After 12 hours of heat treatment, the product was heated to 100° C. and heat-treated for 2 hours to prepare heat-treated lactic acid bacteria (hereinafter referred to as the “heat-killed cells”). The recovered heat-killed cells were then freeze-dried and pulverized to form powder. The total bacterial count of the recovered heat-killed cells was measured using a hemacytometer, and the viable cell count was measured by performing serial dilution, spread-plating onto MRS agar, and incubation at 37° C. for 48 hours.

The above-described preparation of heat-killed cells and bacterial count measurements were performed for each of Lactobacillus plantarum CJLP133, CJLP243, and CJLP55. The total bacterial counts and viable cell counts before (live bacteria) and after (heat-killed bacteria) the heat treatment for each strain are shown in Table 1 below.

TABLE 1

| | | Total Bacterial Count | Viable Cell Count |
|---|---|---|---|
| Strain | Classification | (Cell/g) | (CFU/g) |
| CJLP133 | Live | 1.36E+12 | 2.15E+11 |
| | Heat-killed | 1.36E+12 | 8.23E+03 |
| CJLP243 | Live | 1.43E+12 | 1.14E+12 |
| | Heat-killed | 1.54E+12 | 2.18E+03 |
| CJLP55 | Live | 1.46E+12 | 1.15E+12 |
| | Heat-killed | 1.47E+12 | 9.83E+02 |

As shown in Table 1 above, heat-killed lactic acid bacteria were obtained by reducing the viable cells through the preparation method described above.

### Example 2: Evaluation of the Immunomodulatory Function of Heat-Killed Bacteria

### Example 2-1: Evaluation of the Ability to Promote Interferon-Gamma (IFN-γ) Production

When treating splenocytes of mice biased toward the Th2 response by administration of ovalbumin (OVA) with heat-killed lactic acid bacteria, the ability to promote the production of IFN-γ, a Th1 response-inducing cytokine, was evaluated as follows with reference to Fujiwara et al. (A double-blind trial of Lactobacillus paracasei strain KW3110 administration for immunomodulation in patients with pollen allergy, Allergology International, 2005, Vol. 54, pp. 143-149) and Fujiwara et al. (The antiallergic effects of lactic acid bacteria are strain dependent and mediated by effects on both Th1/Th2 cytokine expression and balance, International Archives of Allergy and Immunology, 2004, Vol. 135, pp. 205-215).

For immunization, five 6-week-old female Balb/c mice were purchased. A mixture was prepared by mixing 1.538 mL of a 13 mg/mL alum hydroxide solution (Sigma), 10 mg of ovalbumin (OVA), and 0.4615 mL of PBS, followed by reaction at room temperature for 20 minutes, and the prepared mixture was intraperitoneally injected into each mouse at a volume of 0.2 mL (containing 1 mg of OVA and 2 mg of alum). The same volume (0.2 mL of the mixture) was administered again intraperitoneally on day 6 for boosting.

On day 13, the mice were sacrificed and the spleens were excised. To each well of a cell culture plate, 100 μL of splenocytes (4×10⁶ cells/mL) obtained from the spleens, 50 μL each of either the live and heat-killed bacteria of the test strains (Lactobacillus plantarum CJLP133, CJLP243, and CJLP55), and 50 μL of OVA (4 mg/mL) were added, followed by incubation in RPMI medium for 5 days in a 10% CO₂ incubator. After incubating for 5 days, the supernatant was collected, and the concentration of IFN-γ was measured by performing an assay using an IFN-γ ELISA kit (Biosource).

The IFN-γ assay using the IFN-γ ELISA kit was conducted according to the kit instructions, and the amount of IFN-γ produced was calculated based on the standard curve of the IFN-γ reference sample provided in the kit, using the O.D. values measured by the ELISA reader.

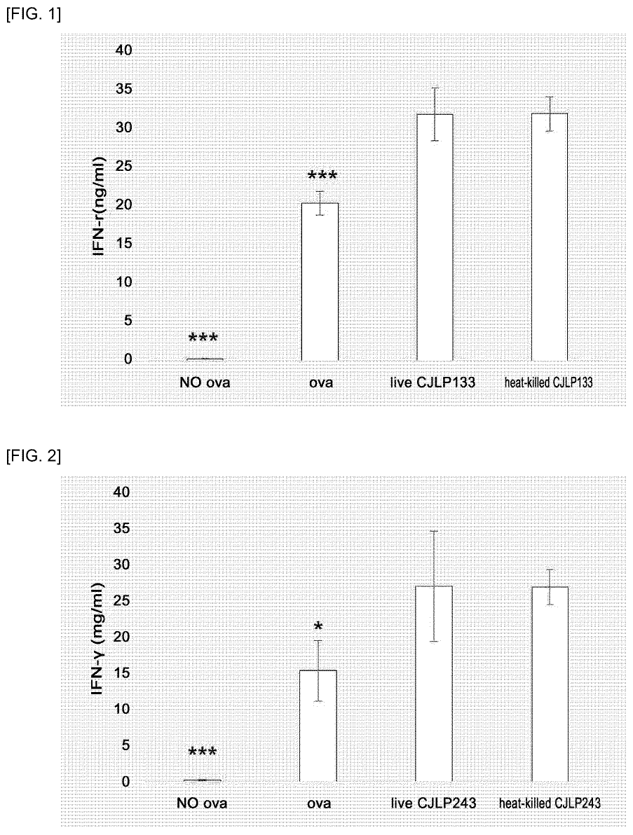

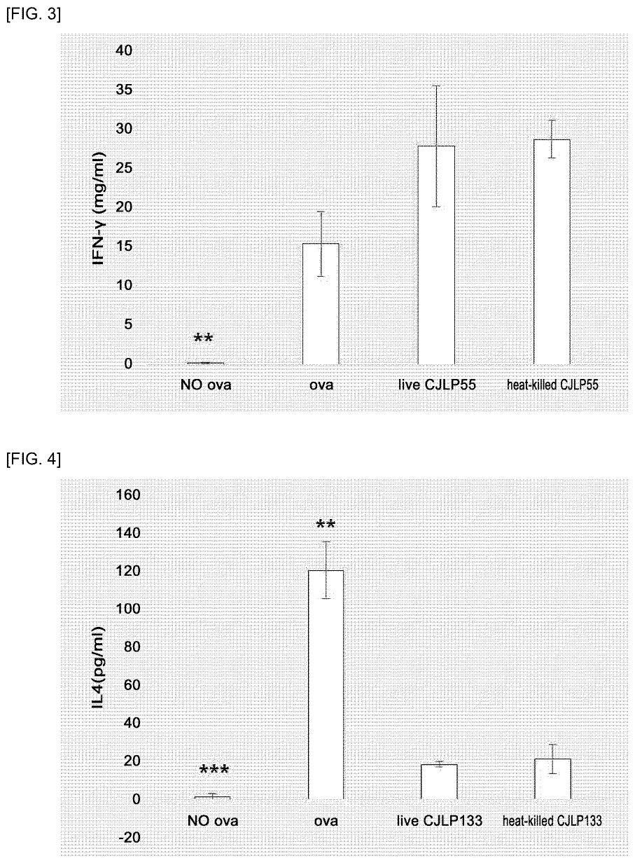

As a result, as shown in FIGS. 1 to 3, IFN-γ significantly increased for both live and heat-killed cells of the CJLP133, CJLP243, and CJLP55 strains as compared to the control group (ova), and the same level of IFN-γ production increase as that of the live bacteria was observed even after heat-killing.

### Example 2-2: Evaluation of the Ability to Reduce Interleukin-4 (IL-4) Production

In order to confirm the inhibitory effect on the production of IL-4, a Th2 response-inducing cytokine, the same experimental conditions as in Example 2-1 were used to treat the splenocytes of mice biased toward Th2 response by OVA administration with heat-killed lactic acid bacteria, except that the IFN-γ ELISA kit was replaced with an IL-4 ELISA kit (Biosource).

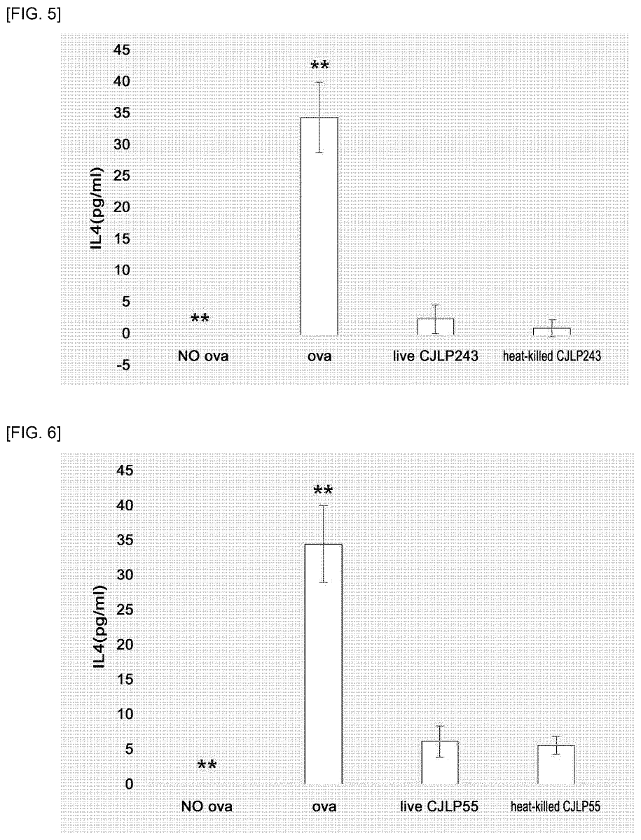

As a result, as shown in FIGS. 4 to 6, IL-4 significantly decreased for both live and heat-killed cells of lactic acid bacteria CJLP133, CJLP243, and CJLP55 as compared to the control group (ova), and the same level of IL-4 production decrease as that of the live bacteria was observed even after heat-killing.

### Example 3: Evaluation of Inhibition of Intestinal Adhesion of Harmful Bacteria (Enterotoxigenic Escherichia coli; ETEC)

The live and heat-killed powder forms of each of the produced lactic acid bacteria strains CJLP133, CJLP243, and CJLP55 were each suspended in physiological saline at a concentration of 1×10⁸ cells/mL. After suspension, centrifugation (14,000 RPM, 4° C., 5 min) was performed to remove the supernatant, followed by resuspension in an equal amount of physiological saline solution for washing. After washing three times in the same manner, the cells obtained by centrifugation were resuspended by dispensing physiological saline in an amount equal to that of the removed supernatant.

The harmful bacteria were cultured in BHI (Brain Heart Infusion) broth medium at 37° C. for 24 hours to measure the total bacterial count, and then diluted with physiological saline to a concentration of 1×10⁸ cells/mL. Subsequently, centrifugation was performed, and the sample was washed three times with physiological saline in the same manner as for the lactic acid bacteria, followed by resuspension by adding physiological saline.

In order to immobilize mucin on a 96-well plate, 100 μL of mucin solution (10 mg mucin/1 mL physiological saline) was dispensed into each well and allowed to stand at 4° C. for 24 hours. After mucin immobilization, each well was washed twice with physiological saline.

Each of the live and heat-killed cells of the prepared lactic acid bacteria (CJLP133, CJLP243, and CJLP55) and the harmful bacterial cell suspension were dispensed into the mucin-coated 96-well plate, and after reacting for 2 hours, the supernatant was removed to eliminate the non-adherent harmful bacteria. After elimination, each well was washed twice with physiological saline, followed by dispensing 100 μL of 0.1% Triton X-100 to recover the cells adhered to the mucin. The inhibitory rate of intestinal adhesion of harmful bacteria was evaluated by analyzing the viable cell count of harmful bacteria adhered before (0 h) and after (2 h incubation) the reaction. For the analysis of the viable cell count of harmful bacteria, the samples were serially diluted using the serial dilution method, spread-plated onto BHI agar, and incubated at 37° C. for 48 hours to measure the bacterial count. The inhibitory rate of intestinal adhesion of harmful bacteria was calculated using the following equation.

$$Inhibitory rate intestinal adhesion ( \% ) = 100 - ( ( Log ( viable cell count at 2 h ) / Log ( viable cell count at 0 h ) ) \times 100 )$$

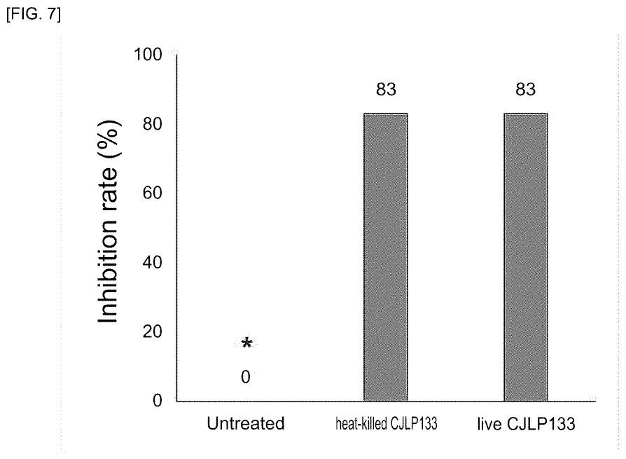

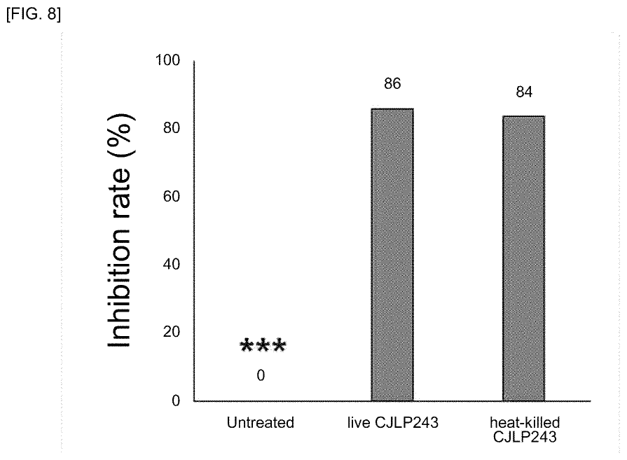

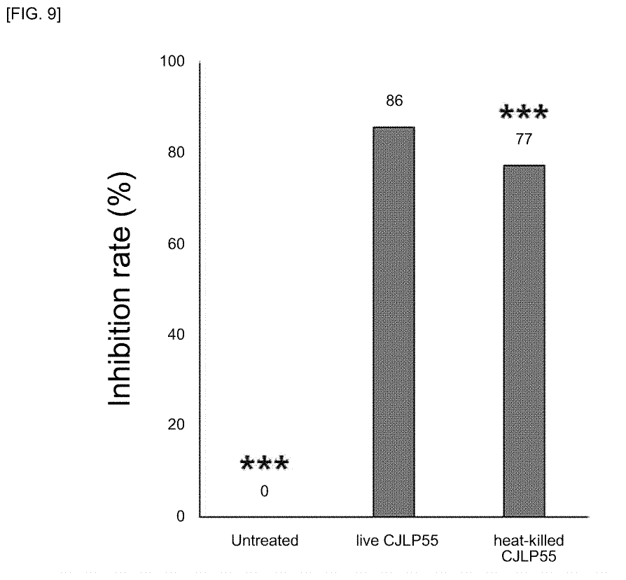

As a result, as shown in FIGS. 7 to 9, the heat-killed cells of the lactic acid bacteria CJLP133, CJLP243, and CJLP55 all exhibited an inhibitory effect on adhesion of harmful bacteria in the intestines at a level equivalent to or similar to that of the viable cells, even after heat-killing.

### Example 4: Evaluation of the Production Levels of IFN-γ and IL-4 and Viable Cell Count of the CJLP133 Strain after First Heat Treatment According to the Temperature and Duration of the Heat Treatment

To evaluate various heat-killing conditions, lactic acid bacteria (CJLP133) were subjected to a first heat treatment under various temperature (40° C., 60° C., 80° C., 100° C., 121° C.) and duration (1 h, 2 h, 4 h, 6 h, 8 h, 10 h, 12 h, 56 h) conditions.

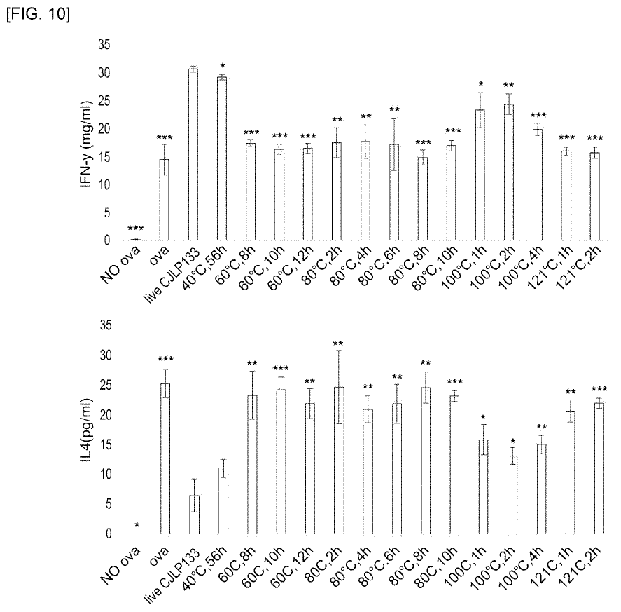

As a result, as shown in FIG. 10, treatment of CJLP133 cells at 40° C. for 56 h and 100° C. for 2 h exhibited higher IFN-γ production and lower IL-4 production compared to other temperatures, with the highest IFN-γ production and the lowest IL-4 production observed at 40° C. for 56 h.

Meanwhile, the results of viable cell count measurements under various temperature and duration conditions are shown in Table 2 below.

TABLE 2

| Temperature (° C.) | Duration (h) | CFU/mL |
|---|---|---|
| 40 | 56 | 6.2.E+06 |
| 60 | 6 | 4.9.E+01 |
| | 8 | 1.9.E+01 |
| | 10 | 1.7.E+01 |
| | 12 | 2.0.E+02 |
| 80 | 2 | 7.7.E+02 |
| | 4 | 1.1.E+01 |
| | 6 | 4.9.E+00 |
| | 8 | 1.1.E+00 |
| | 10 | 1.1.E+03 |
| 100 | 1 | 2.7.E+07 |
| | 2 | 4.9.E+00 |
| | 4 | 4.9.E+00 |
| 121 | 1 | 1.5.E+04 |
| | 2 | 2.7.E+05 |

As a result, as shown in Table 2, viable cells were generally present at levels below the reference value (1×10⁴ CFU/g or less) under most temperature conditions; however, in the case of treatment at 40° C. alone, viable cells remained at a level higher than the reference value.

### Example 5: Evaluation of the Production Levels of IFN-γ and IL-4 and Viable Cell Count after Single Heat Treatment (First Heat Treatment) and Double Heat Treatment (First and Second Heat Treatments)

In order to compare heat-killing between single heat treatment (first heat treatment) and double heat treatment (first and second heat treatments), the amounts of IFN-γ and IL-4 produced were evaluated for heat-killed lactic acid bacteria (CJLP133) subjected to single or double heat treatment at 40° C. for various durations.

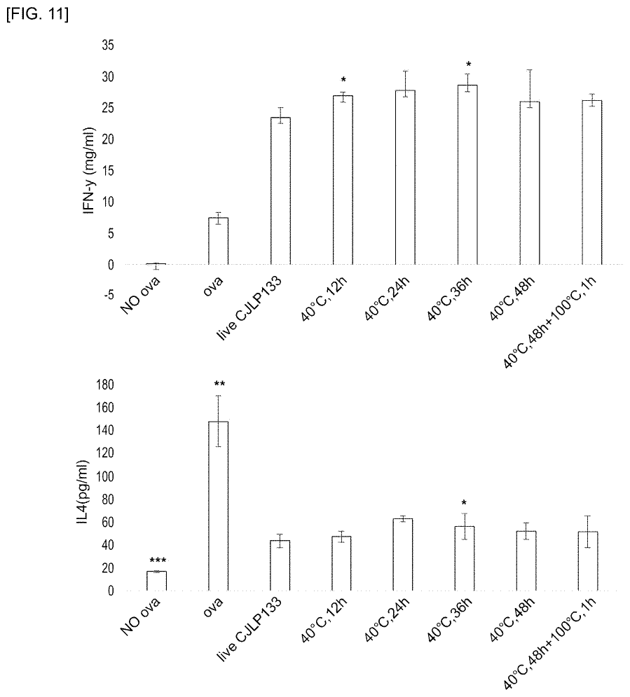

As a result, as shown in FIG. 11, when the first heat treatment was performed at 40° C. for 12 hours or more, the levels of IFN-γ and IL-4 production were found to be at the same level as those of the live cells. In addition, even when an additional heat treatment (second heat treatment) at 100° C. for 1 hour was applied, the levels of IFN-γ and IL-4 production were found to be at the same level as those of the live cells.

Meanwhile, the results of viable cell count measurements under the same heat treatment conditions are shown in Table 3 below.

TABLE 3

| First Heat Treatment | Second Heat Treatment | CFU/g |
|---|---|---|
| 40° C., 12 h | — | 4.58E+09 |
| 40° C., 24 h | — | 2.12E+08 |
| 40° C., 36 h | — | 1.44E+08 |
| 40° C., 48 h | — | 1.43E+08 |
| 40° C., 48 h | 100° C. 1 h | 7.7.E+02 |

As a result, as shown in Table 3, residual viable cells were found to be below the reference value (2.4×10³ CFU/g) after the first and second heat treatments.

### Example 6: Evaluation of the Production Levels of IFN-γ and IL-4 and Viable Cell Count According to Temperature and Duration Upon Combined Application of Double Heat Treatment (First and Second Heat Treatments)

In order to evaluate various double heat treatments (first and second heat treatments), the levels of IFN-γ and IL-4 production were assessed for heat-killed lactic acid bacteria (CJLP133) subjected to the first heat treatment at 40° C. for varying durations alone, and to both the first and second heat treatments.

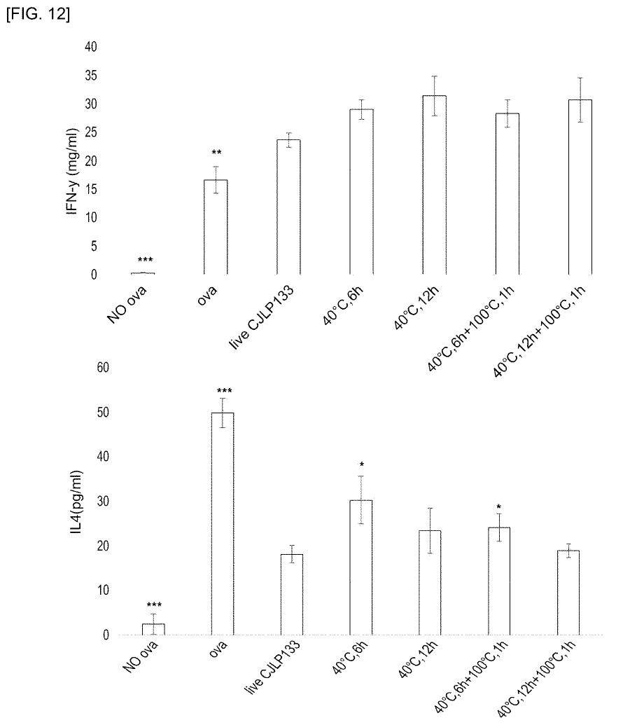

As a result, as shown in FIG. 12, regardless of whether the second heat treatment was applied, it was confirmed that IFN-γ production increased and IL-4 production decreased when the first heat treatment was performed at 40° C. for 12 hours, compared to the first heat treatment at 40° C. for 6 hours. In addition, it was confirmed that even when the second heat treatment at 100° C. was additionally applied following the first heat treatment at 40° C. for 6 hours and the first heat treatment at 40° C. for 12 hours, results similar to those of the first heat treatments alone were observed.

Meanwhile, the results of viable cell count measurements under the same heat treatment conditions are shown in Table 4 below.

TABLE 4

| First Heat Treatment | Second Heat Treatment | CFU/g |
|---|---|---|
| 40° C., 6 h | — | 8.89E+09 |
| 40° C., 12 h | — | 4.22E+09 |
| 40° C., 6 h | 100° C., 1 h | 2.22E+04 |
| 40° C., 12 h | 100° C., 1 h | 1.22E+04 |

As a result, as shown in Table 4, it was confirmed that the viable cell count decreased when the second heat treatment at 100° C. was additionally applied, compared to the first heat treatment at 40° C. alone.

### Example 7: Evaluation of the Production Levels of IFN-γ and IL-4 and Viable Cell Count of Other Lactic Acid Bacteria According to the Temperature and Duration of the Heat Treatment

To evaluate various heat-killing conditions for the lactic acid bacteria CJLP243 and CJLP55, each strain was subjected to a single heat treatment (first heat treatment) under various temperature (40° C., 60° C., 80° C., 100° C., 121° C.) and duration (1 h, 2 h, 4 h, 6 h, 8 h, 10 h, 12 h, 24 h) conditions.

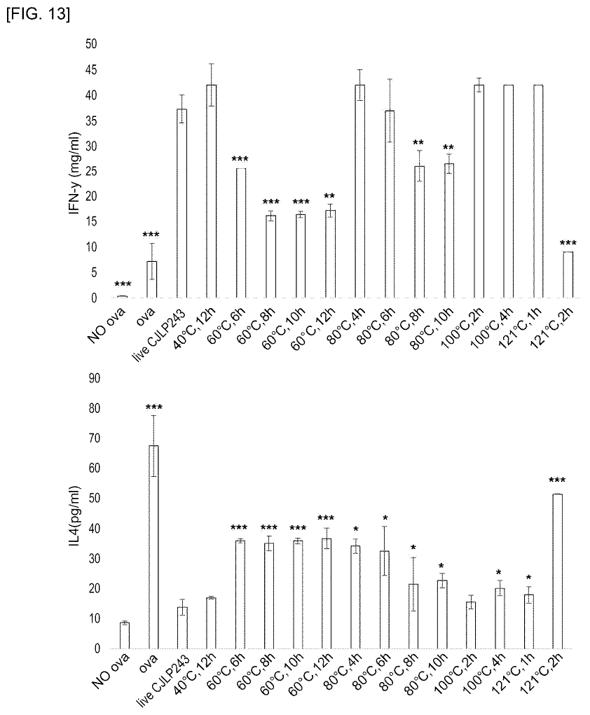

As a result, as shown in FIG. 13, treatment of CJLP243 cells at 40° C. for 12 hours and 100° C. for 2 hours exhibited higher IFN-γ production and lower IL-4 production compared to other temperature conditions.

Meanwhile, the results of viable cell count measurements for CJLP243 under the same heat treatment conditions are shown in Table 5 below.

TABLE 5

| Temperature (° C.) | Duration (h) | CFU/g |
|---|---|---|
| 40 | 12 | 1.9.E+10 |
| 60 | 6 | 5.9.E+02 |
| | 8 | 1.2.E+03 |
| | 10 | 2.1.E+03 |
| | 12 | 1.6.E+03 |
| 80 | 4 | 2.7.E+03 |
| | 6 | 1.3.E+02 |
| | 8 | 3.8.E+04 |
| | 10 | 1.1.E+02 |
| 100 | 2 | 8.6.E+04 |
| | 4 | 1.1.E+03 |
| 121 | 1 | 2.6.E+03 |
| | 2 | 2.3.E+02 |

As a result, as shown in Table 5 above, the number of viable cells was generally lower than the standard (1×10⁴ CFU/g or less) at most temperatures, but when treated alone at 40° C., the number of viable cells remained higher than the standard.

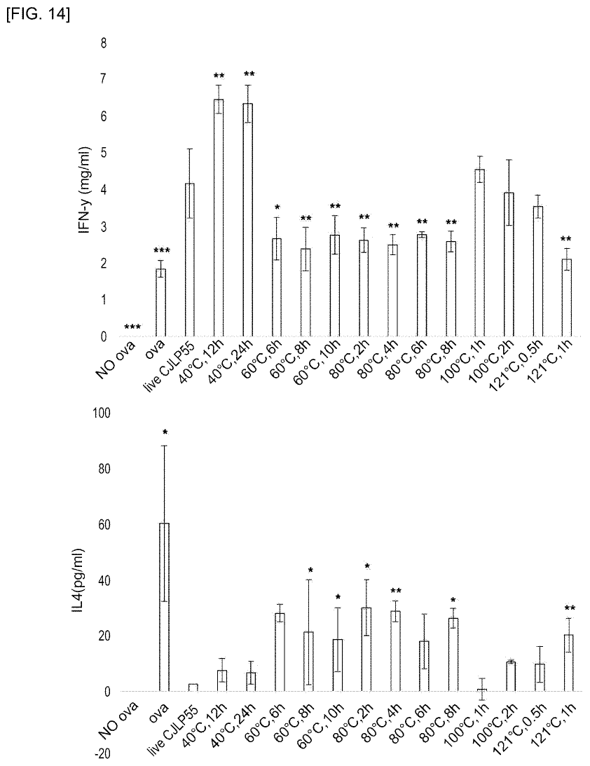

In addition, as shown in FIG. 14, the treatment of CJLP55 cells at 40° C. for 12 hours and 100° C. for 2 hours exhibited higher IFN-γ production and lower IL-4 production compared to other temperature conditions.

Meanwhile, the results of viable cell count measurements for CJLP55 under the same heat treatment conditions are shown in Table 6 below.

TABLE 6

| Temperature (° C.) | Duration (h) | CFU/g |
|---|---|---|
| 40 | 12 | 2.2.E+10 |
| | 24 | 2.0.E+07 |
| 60 | 6 | 3.4.E+03 |
| | 8 | 9.5.E+02 |
| | 10 | 6.0.E+02 |
| 80 | 2 | 3.2.E+03 |
| | 4 | 5.0.E+02 |
| | 6 | 1.2.E+03 |
| | 8 | 5.5.E+03 |
| 100 | 1 | 6.0.E+02 |
| | 2 | 1.9.E+03 |
| 121 | 0.5 | 4.9.E+05 |
| | 1 | 1.9.E+03 |

As shown in Table 6 above, in general, the number of viable cells was lower than the standard (1×10⁴ CFU/g or less) at other temperatures, but when treated at 40° C. alone, the number of viable cells remained higher than the standard.

The above results suggest that the lactic acid bacteria CJLP243 and CJLP55 exhibit results similar to those of CJLP133.

From the above results, it was confirmed that the method for preparing heat-killed lactic acid bacteria comprising the first and second heat treatments of the present disclosure can significantly enhance the immunomodulatory ability of the lactic acid bacteria.

As set forth above, a person skilled in the art will be able to understand that the present disclosure may be embodied in other specific forms without departing from the technical spirit or essential characteristics thereof. Therefore, the embodiments described above should be construed as being exemplified and not limiting the present disclosure. It should be understood that all changes or modifications derived from the definitions and the scopes of the claims and their equivalents fall within the scope of the present disclosure.

1. A method for preparing heat-killed lactic acid bacteria, comprising: a first heat treatment step of heat-treating one or more lactic acid bacteria selected from the group consisting of Lactobacillus plantarum CJLP133 deposited under Accession Number KCTC 11403BP, Lactobacillus plantarum CJLP243 deposited under Accession Number KCCM 11045P, and Lactobacillus plantarum CJLP55 deposited under Accession Number KCTC 11401BP at a temperature of 40° C. or more and less than 60° C. for 6 to 56 hours; and
a second heat treatment step of heat-treating the lactic acid bacteria at a temperature of 90° C. to 125° C. for 10 minutes to 4 hours.

2. The method according to claim 1, wherein the method increases the immunomodulatory ability of the heat-killed lactic acid bacteria.

3. The method according to claim 1, wherein the method increases an ability of the lactic acid bacteria to promote production of interferon-gamma (IFN-γ), reduce production of interleukin-4 (IL-4), or inhibit adhesion of harmful bacteria in the intestines, or a combination thereof.

4. The method according to claim 1, wherein the first heat treatment step is heat-treating lactic acid bacteria at a temperature of 40° C. to 50° C. for 6 to 24 hours.

5. The method according to claim 1, wherein the second heat treatment step is heat-treating lactic acid bacteria at a temperature of 90° C. to 121° C. for 1 to 4 hours.

6-8. (canceled)

9. Heat-killed lactic acid bacteria prepared by the method of claim 1.

10. A composition comprising the heat-killed lactic acid bacteria of claim 9 wherein the composition is for a food composition, a pharmaceutical composition or a feed composition.

11. The food composition according to claim 10, wherein the food composition is for immunomodulation.

12-13. (canceled)
